(19) United States
(12) Patent Application Publication
(10) Pub. No.: US 2007/0112287 A1
(43) Pub. Date: May 17, 2007

(54) SYSTEM AND METHOD FOR DETECTING DEVIATIONS IN NOMINAL GAIT PATTERNS

(76) Inventors: Craig L. Fancourt, Lawrenceville, NJ (US); John Gregory Aceti, Lexington, MA (US)

Correspondence Address: PATENT DOCKET ADMINISTRATOR;LOWENSTEIN SANDLER P.C., 65 LIVINGSTON AVENUE, ROSELAND, NJ, 07068, US

(21) Appl. No.: 11/531,530

(22) Filed: Sep. 13, 2006

Related U.S. Application Data
(60) Provisional application No. 60/716,624, filed on Sep. 13, 2005.

(51) Int. Cl. A61B 5/103; G08B 23/00

(52) U.S. Cl. 600/595; 340/573.7

(57) ABSTRACT

A monitoring device having a system and method for detecting deviations in nominal gait patterns is provided. The monitoring device is configured for at least partial insertion within an auditory canal of an individual. The system employed in the monitoring device is comprised of at least an accelerometer configured for measuring acceleration in three orthogonal directions aligned with the principal directions of the body. Gait features values are extracted from the measurements and continuously accumulated, thereby establishing the nominal gait pattern of the individual. Subsequent extractions of current gait features are compared to accumulated gait feature statistics to determine if a deviation in the established nominal gait pattern has been detected.

### CROSS-REFERENCE TO RELATED APPLICATIONS

This application is a continuation-in-part of pending U.S. patent application Ser. No. 10/925,765, filed Aug. 25, 2004, and additionally claims the benefit of U.S. Provisional Patent Application No. 60/716,624, filed Sep. 13, 2005.

### BACKGROUND OF THE INVENTION

1. Field of the Invention

The present invention relates to the field of medical monitoring. More particularly, the present invention is directed to a system and method for detecting deviations in nominal gait patterns by employing a monitoring device adaptable for at least partial insertion within or, alternatively, for attachment in close proximity to the auditory canal.

2. Description of the Prior Art

The manner in which an individual traverses movements on foot is referred to as gait. The most basic of human gaits are walking and running. Such basic movements are regulated by the human body's neuromuscular and musculoskeletal systems. Through the combined coordination of muscle contractions, joint movements and sensory perception, an individual may traverse a path of travel in any desirable manner.

Assessing an individual's gait pattern can be useful for identifying particular areas of impairment and neurological defects affecting motor control. For example, information pertaining to an individual's particular gait pattern may generally be used to determine the existence of a physical impairment associated with weakened muscle development, limited range in the movement of joints or poor posture. Typically, an individual's gait pattern is assessed in connection with pathological conditions such as cerebral palsy, multiple sclerosis, Parkinson's disease, frontal lobe disease and various other neuromuscular disorders. However, providing a means for assessing an individual's gait pattern is not only useful for identifying physical impairments and neurological diseases, but rather it is also tremendously useful for monitoring the progress of rehabilitative measures and the recovery of patients after, for example, an injury resulting in bone fractures or orthopedic related surgical procedures. When properly assessed, an individual's particular gait pattern can help guide caregivers in determining the appropriate treatments and preventative measures to be implemented.

Gait analysis entails the process of quantifying and interpreting an individual's manner of movement. Modern means for analyzing gait have been known to employ a variety of sophisticated equipment. For instance, one commonly employed means for analyzing an individual's particular gait involves the use of video cameras positioned around a walkway having markers designating anatomical landmark points. The video cameras record the individual's movements as he/she traverses the walkway, wherein the recorded movements are then applied to a computer model for determining the underlying gait related motions of the individual. Other means for monitoring gait have employed the use of multiple wearable body sensors strategically positioned and attached to the upper torso of an individual, such as those described in U.S. Pat. No. 5,919,149, for diagnosing stability and balance impairments resulting from disorders.

These aforementioned gait assessment techniques, although non-invasive and likely effective in assessing the gait of an individual, require the use of expensive and intricate equipment set-ups. In addition, they are deficient in that they limit the extent of medically related applications in which gait assessment can be made useful. A lab type environment is typically required due to the sophistication and intricacies of these gait assessment techniques and, therefore, there are obvious limitations on the scope for which these gait assessment techniques can be used. For example, it would very difficult and costly to provide the aforementioned gait assessment techniques as a means for allowing continuous monitoring of individuals undergoing recovery. Existing gait assessment techniques lack ease of mobility. Moreover, every individual possesses a unique gait. In fact, gait is actively studied as a potential biometric marker For this reason, it is difficult to build an absolute gait classifier to indicate whether a particular gait is normal or pathological. Because the range of human gaits is so expansive, what may be a normal gait for one individual may be indicative of a health problem for another.

Accordingly, there exists a need for an improved system and method that can be employed through use of a monitoring device that is minimally invasive and maximizes mobility to allow for the detection of a deviation in an individual's unique nominal gait pattern.

### SUMMARY OF THE INVENTION

It is an object of the present invention to provide a minimally invasive monitoring device employing a light weight and cost effective design, thereby further providing a less cumbersome and highly mobile means for monitoring an individual's gait pattern.

It is another object of the present invention to provide a minimally invasive and mobile monitoring device capable of providing continuous monitoring of an individual's gait pattern to evaluate the effectiveness of rehabilitative measures, drug efficacy and restricted movements by an individual undergoing recovery.

It is another object of the present invention to provide a minimally invasive and mobile monitoring device capable of detecting various deviations in the unique nominal gait pattern of an individual under surveillance, thereby identifying early signs of neurological problems, deterioration rate of motor control and warning signs of imminent falls.

These and other objects are accomplished in accordance with the principles of the present invention, wherein the novelty of the present invention will become apparent ftom the following detailed description and appended claims.

In accordance with the present invention, a monitoring device configured for at least partial insertion within the auditory canal of an individual is provided. The monitoring device employs a system and method for detecting a deviation in a nominal gait pattern through use of an accelerometer embodied therein and positioned securely within an individual's auditory canal. In an alternate embodiment, the accelerometer is positioned in close proximity to the auditory canal by securely attaching the monitoring device between an auricle of an ear and a head of the individual. The accelerometer measures acceleration in three orthogonal directions aligned with the principal directions of the body. Acceleration measurements are digitized and processed through use of a Kalman filter, providing an estimation of the current state of the body. Gait features associated with the current state of the body are continuously extracted and accumulated to determine the unique nominal gait pattern of the individual. As new gait features associated with a current state of the body are extracted they are compared with previously accumulated gait feature statistics to detect deviations in the established nominal gait pattern of the individual. Suitable notification procedures are subsequently executed in response to detected deviations in the nominal gait pattern.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a block diagram depicted within an exemplary monitoring device suitable for insertion within the auditory canal in accordance with a preferred embodiment of the present invention.

FIGS. 2A and 2B are illustrations of an exemplary monitoring device suitable for placement between an auricle of an ear and a head in accordance with another embodiment of the present invention.

FIG. 3 is a flowchart illustrating the steps employed in detecting a deviation in a nominal gait pattern in accordance with a preferred embodiment of the present invention.

It is to be understood that the above-identified drawing figures are for purposes of illustrating the concepts of the present invention and may not be to scale, and are not intended to be limiting in terms of the range of possible shapes and proportions of the present invention.

### DETAILED DESCRIPTION OF THE PREFERRED EMBODIMENTS

The present invention is directed towards a system and method for detecting a deviation from an individual's nominal gait pattern. The present invention also serves as a means for providing continuous medical monitoring. For purposes of clarity, and not by way of limitation, illustrative views of the present invention are described with references made to the above-identified figures. Various modifications obvious to one skilled in the art are deemed to be within the spirit and scope of the present invention.

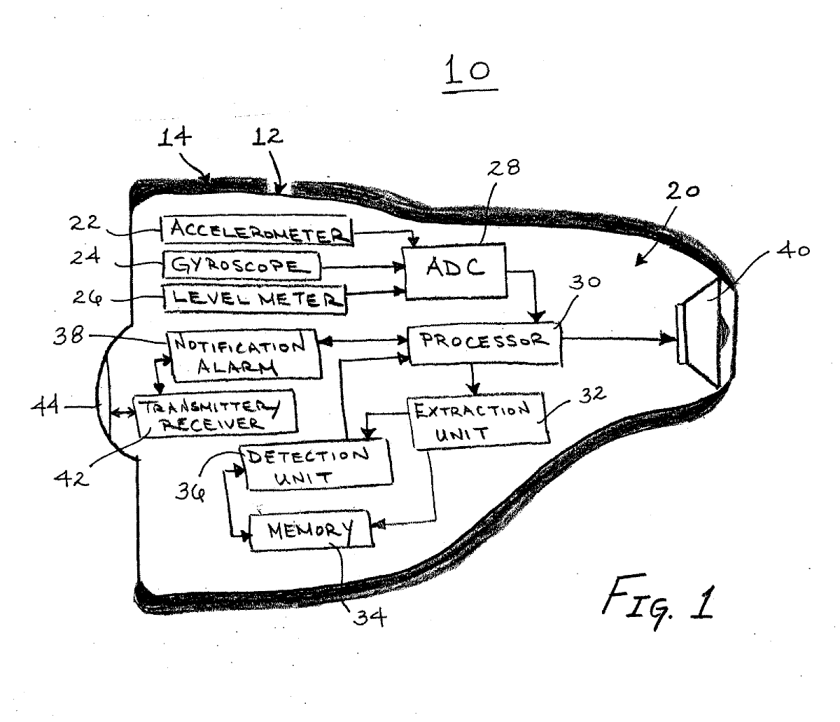

An exemplary monitoring device 10 is illustrated in FIG. 1. In accordance with a preferred embodiment of the present invention, monitoring device 10 is constructed and configured for at least partial insertion within an auditory canal of an individual. Monitoring device 10 is comprised of a housing 12 having gait change detection system 20 enclosed therein. A moldable exterior shell 14 may be provided circumferentially about the exterior surface of housing 12 of monitoring device 10. Moldable exterior shell 14 is preferably constructed of a soft, yet durable, material capable of conforming to the interior walls of an individual's auditory canal in order to provide a comfortable and secure fitting of monitoring device 10. For example, moldable exterior shell 14 may be constructed of a memory foam that can be compressed and inserted into the auditory canal. When the memory foam is released it expands and provides a secure custom fitting within the individual's auditory canal. It will be understood that the use of a memory foam is one of many suitable materials that may be used to construct a moldable exterior shell 14 and is merely provided as an example.

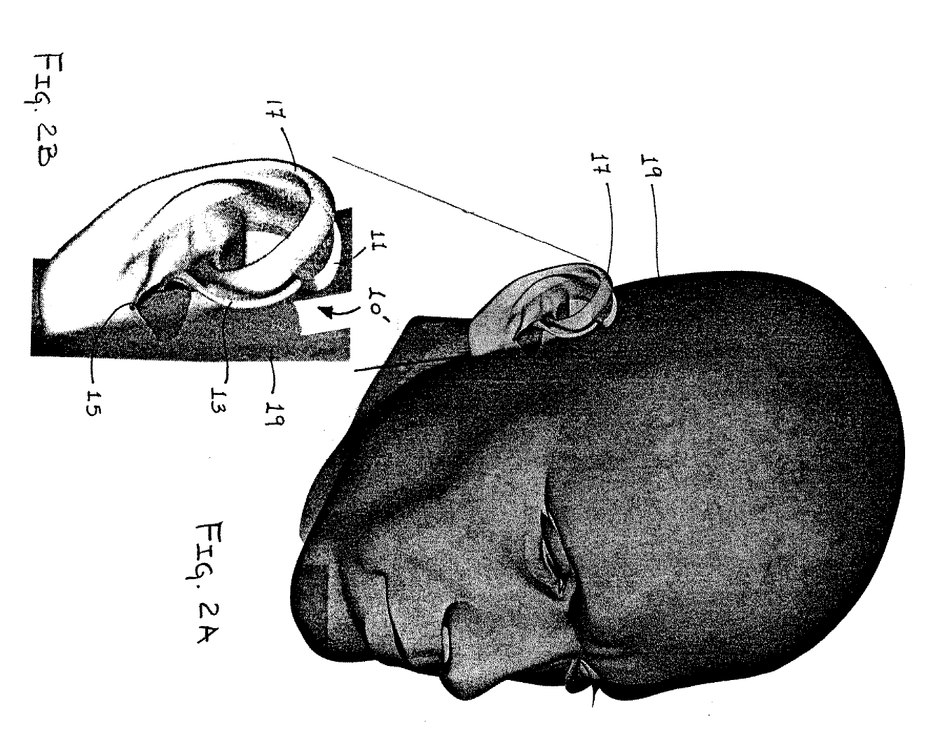

In an alternative embodiment, monitoring device 10 of FIG. 1 may be constructed so as to be situated only partially within the auditory canal of an individual. For example, as illustrated in FIGS. 2A and 2B, an exemplary monitoring device 10′ is comprised of a housing 11, a processing extension 13 and a plug 15. Monitoring device 10′ is configured for suitable placement between an auricle 17 of an ear and a head 19 of an individual. As illustrated in the enlarged view of FIG. 2B, housing 11 is shaped to the curved contour of the ear of an individual. Processing extension 13 extends from an end of housing 11 to plug 15, which is inserted within the auditory canal of the individual. Plug 15 may similarly be constructed with a moldable exterior shell 14 (not shown) to provide a secure custom fitting within the auditory canal of the individual.

Gait change detection system 20 provided within housing 12 of FIG. 1 is comprised of at least one accelerometer 22, an analog-to-digital (ADC) converter 28, a processor 30, an extraction unit 32, a memory component 34, a detection unit 36, a notification alarm 38, a speaker 40, a wireless transmitter/receiver 42, and an antenna 44. The components of system 20 may, similarly, be provided entirely within housing 11 (not shown) or, alternatively, distributed between housing 11 and plug 15 and connected by processing extension 13 of FIG. 2B. For example, accelerometer 22 may be fixed within bud 15, wherein corresponding signals are transmitted to ADC 28 and processor 30 provided in housing 11 via processing extension 13.

Various neurological diseases that affect motor control and gait possess unique identifiable characteristics. For example, in frontal lobe disease an individual is prone to small shuffling steps. These small shuffling steps might be detected from features such as forward velocity, vertical acceleration and step frequency. As another example, in Parkinson's disease an individual is prone to small rapid steps, small backward steps after attempting to stop and difficultly turning quickly. The small rapid steps and backward steps are also detectable as a signature identifier of this particular disease. The progression of neurological diseases that affect motor control, as well as imminent dangers of falling due to dizziness, vertigo or blackout, can be detected simply by employing a monitoring means for assessing the distinguishable random direction and step frequencies associated with each. Therefore, accelerometer 22 is preferably a three-axis accelerometer that can be used to measure acceleration in the three orthogonal directions aligned with the principal directions of the human body.

In order for accelerometer 22 to be useful in monitoring an individual's gait, it must be of the type sensitive to low frequency and low amplitude motions. In addition, accelerometer 22 is preferably of small circuit design and low power consumption, such as those produced by Hitachi Metals, Ltd., so that it may be properly integrated within the limited space allotted by housing 22. A gyroscope 24 and a level meter 26 may also be integrated into system 20 to supplement the acceleration measurements taken by accelerometer 22. A gyroscope is typically used to measure and maintain orientation. For example, gyroscope 24 may be integrated in conjunction with accelerometer 22 of system 20 to correct for horizontal motions of an individual's head not related to body movement. Similarly, level meter 26 may be provided to correct for vertical motions of the head not related to body movement.

Accelerometer 22, gyroscope 24 and level meter 26 are coupled to processor 30 through ADC 28. Acceleration signals measured by accelerometer 22, as well as corrective measurements provided by gyroscope 24 and level meter 26, are digitized by ADC 28 after any necessary signal conditioning and pre-processing. Processor 30 is further coupled to extraction unit 32 and detection unit 36 in order to, respectively, extract and analyze relevant gait features corresponding to the acceleration measurements provided by accelerometer 22. Relevant gait features are continuously extracted and may be stored in memory component 34 in order to accumulate gait feature statistics and derive the nominal gait pattern of a particular individual.

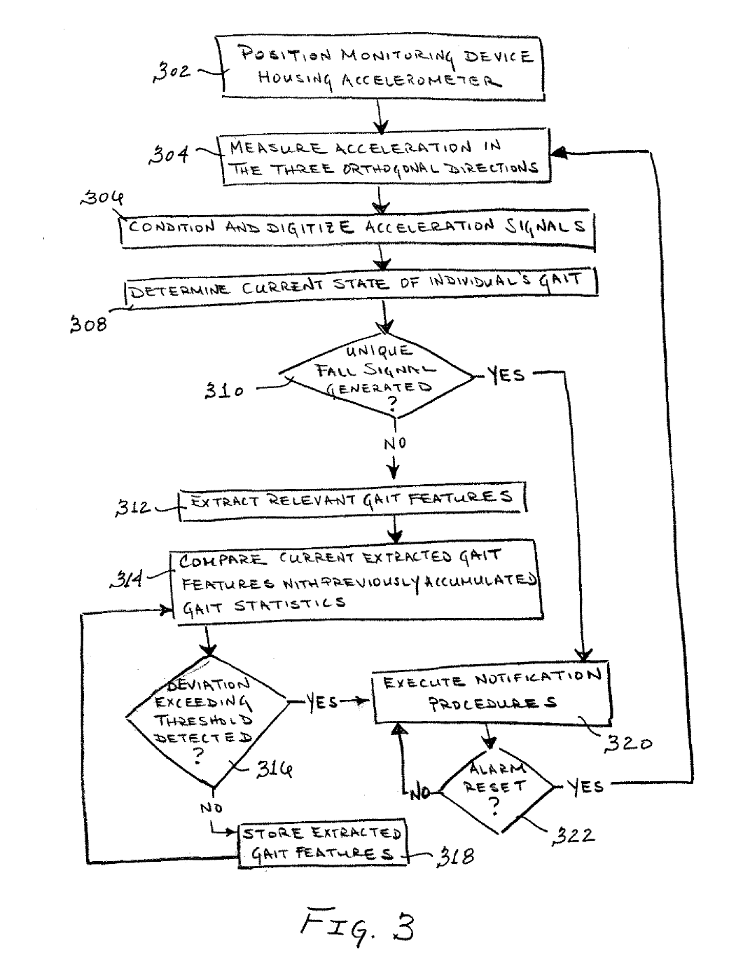

FIG. 3 is an illustrative depiction of the steps employed by system 20 for monitoring and detecting a deviation in an individual's nominal gait pattern. System 20 is initiated, at step 302, after proper and secure positioning of monitoring device 10 or housing 11 and plug 15 of monitoring device 10′, respectively, within the auditory canal or between the auricle 17 and head 19 of an individual. Upon the proper positioning of monitoring device 10 or 10′ at step 302, system 20 is activated and acceleration is measured, at step 304, in the three orthogonal directions aligned with the principal directions of the individual's body. Necessary conditioning, pre-processing and digitizing of the acceleration signals measured by accelerometer 22 are then performed at step 306. After acceleration signals measured by accelerometer 22 are digitized at step 306, the digitized acceleration signals are processed by processor 30 at step 308. Processor 30 may employ a Kalman filter in conjunction with the digitized acceleration measurements to derive estimations of the current state of the individual, comprising of the individual's position, velocity and acceleration in three axes at a particular point in time. Thereafter, relevant gait features are extracted from the estimations at step 312.

In some instances, specific gait feature may be predefined in system 20 and associated with an indication that an individual has fallen or is in clear imminent danger of falling. When these predefined gait feature values are identified, system 20 may be configured to bypass other processing procedures and automatically trigger notification procedures to alert a third party that an individual under surveillance has fallen or is on the verge of an imminent fall. The notification procedures may also be configured to warn the individual of a potential loss of balance and an imminent fall. Therefore, if a gait feature value extracted from current state estimations generated at processing step 308 matches a predefined gait feature value, or is classified within a predefined range of values identified at step 310, system 20 may bypass intermediate steps 314 and 316 (discussed in further detail below) and execute notification procedures in connection with step 320.

If predefined gait feature values, as described above, are not matched at step 310, system 20 proceeds with the execution of step 312. At step 312, relevant gait features are extracted from the current state readings provided at processing step 308, generating a time-to-space mapping of locally stationary relevant gait features that have been extracted. Statistics of these relevant gait features are accumulated over a period of time and stored, for example, in memory component 34 of system 20. Alternatively, for substantially longer periods of time involving the monitoring of an individual, statistics of relevant gait features may be transmitted via wireless transmitter/receiver 42 employing antenna 44 to a remote monitoring location (not shown) for storage and analysis.

As relevant gait features are extracted from current state estimations and accumulated into memory, system 20 is continuously comparing the values associated with current state gait features to previously accumulated gait features statistics, as provided in step 314. This continuous comparison and accumulation of gait feature statistics permits system 20 to determine the nominal gait pattern for the particular individual under surveillance. Once an nominal gait pattern is recognizable, system 20 becomes highly efficient in detecting deviations exceeding a predefined permissible range. Deviation detection is performed at step 316 using, for example, detection unit 36 of FIG. 1. Current gait feature values acquired by extraction unit 32 and previously accumulated gait feature statistics stored in memory component 34 are made accessible to detection unit 36, allowing detection unit 36 to execute a comparison and determine whether there is a deviation exceeding a predefined permissible threshold.

If a deviation is not detected, currently extracted gait features are accumulated with previously extracted gait features at step 318, furthering contributing to the derivation of the nominal gait pattern of the individual under surveillance. The accumulation of gait features stored at step 318 is linked back to step 314 to allow for ongoing comparison of incoming gait features with previously accumulated gait features statistics. However, if a deviation is detected at step 316, detection unit 36 of system 20 may report the detected deviation back to processor 30, triggering the execution of notification procedures at step 320. System 20 may be configured so that notification procedures executed at step 320 may continue to be executed until the particular notification alarm is reset at step 322. Once the notification alarm is reset, system 20 may return to step 304 to continue measuring acceleration of the individual under surveillance.

Notification procedures executed at step 320 may include the transmission of a notification alarm to the caregiver and an audible alarm or a predefined notification message to the individual under surveillance. For example, in the case of detecting an imminent fall, an audible alarm may be sounded to the patient via speaker 40 of monitoring device 10, as well as to the caregiver via a wireless transmission using transmitter/receiver 42 coupled to antenna 44 of monitoring device 10. The notification procedures executed at step 320 may also include the transmission of a notification message by a caregiver from a remote location to the individual under surveillance upon receiving a notification alarm indicating a deviation in the individual's nominal gait pattern or, alternatively, triggering of a predefined notification message stored in memory component 34 of system 20 to be aurally presented to the individual via speaker 40 of monitoring device 10. For example, a notification message may be wirelessly transmitted by the caregiver from a remote location to system 20 of monitoring device 10 or 10′ upon receipt of a notification alarm and aurally presented to the individual under surveillance. It should be understood that the notification procedures described above are provided merely as examples and that various notification procedures may be implemented in accordance with the principles of the invention.

One skilled in the art will appreciate that the present invention can be practiced by other than the described embodiments, which are presented for purposes of illustration and not by way of limitation, and the present invention is limited only by the claims that follow.

What is claimed is:

1. A system for determining a deviation in a nominal gait pattern of an individual, comprising:
a multi-axis accelerometer;
a processor coupled to said multi-axis accelerometer for analyzing measurements collected by said multi-axis accelerometer;
an extraction component coupled to said processor for extracting current gait features associated with measurements analyzed by said processor;
a memory component for storing extracted gait feature statistics; and
a detection component coupled to said extraction component and said processor for comparing said current gait features to extracted gait feature statistics accumulated in said memory component.

2. The system of claim 1, wherein said multi-axis accelerometer, said processor, said extraction component and said detection component are provided within a housing made suitable for at least partial insertion within an auditory canal of said individual.

3. The system of claim 1, wherein said multi-axis accelerometer, said processor, said extraction component and said detection component are provided within a housing made suitable for attachment between an auricle of an ear and a head of said individual.

4. The system of claim 1, wherein said multi-axis accelerometer, said processor, said extraction component and said detection component are distributed between a housing and a plug, wherein said housing is attached between an auricle of an ear and a head, and wherein said plug is suitable for at least partial insertion within an auditory canal of said individual.

5. The system of claim 4, wherein said housing and said plug are coupled together via a processing extension, said processing extension providing an electrical connection between said housing and said plug.

6. The system of claim 5, wherein said plug is configured to accommodate said multi-axis accelerometer to be securely positioned within said auditory canal of said individual.

7. The system of claim 1, wherein said multi-axis accelerometer measures acceleration in three orthogonal directions aligned with principal directions of said individual's body.

8. The system of claim 1, wherein said processor employs a Kalman filter for estimating position, velocity and acceleration associated with a current state of said individual's body.

9. The system of claim 1, further comprising a gyroscope coupled to said processor.

10. The system of claim 9, wherein said gyroscope supplements multi-axis accelerometer measurements by providing corrective measurements in connection with horizontal motions of a head unrelated to body movement.

11. The system of claim 1, further comprising a level meter coupled to said processor.

12. The system of claim 11, wherein said level meter supplements multi-axis accelerometer measurements by providing corrective measurements in connection with vertical motions of a head unrelated to body movement.

13. The system of claim 1, wherein said nominal gait pattern is derived by evaluating said extracted gait feature statistics accumulated in said memory component.

14. The system of claim 1, wherein said detection component determines said deviation in said nominal gait pattern when values of said current gait features exceed permissible threshold values established by said extracted gait feature statistics accumulated in said memory component.

15. The system of claim 1, further comprising a notification component having a means for wireless transmission of data associated with said extraction and said detection components.

16. The system of claim 15, wherein said notification component is configured to communicate an alert signal when said deviation in said nominal gait pattern has been detected.

17. The system of claim 16, wherein said alert signal is an audible tone.

18. The system of claim 16, wherein said alert signal is a predefined notification message.

19. A method for determining a deviation in a nominal gait pattern of an individual, comprising:
measuring acceleration in three orthogonal directions aligned with principal directions of said individual's body;
estimating position, velocity and acceleration of said individual's body;
extracting current gait features associated with said estimations; and
comparing said extracted current gait features with stored gait feature statistics to determine the existence of said deviation.

20. The method of claim 19, wherein said measuring acceleration in three orthogonal directions further comprises securely positioning a multi-axis accelerometer within an auditory canal of said individual.

21. The method of claim 19, wherein said measuring acceleration in three orthogonal directions further comprises securely attaching a multi-axis accelerometer between an auricle of an ear and a head of said individual.

22. The method of claim 19, wherein said estimations of said position, velocity and acceleration of said individual's body are determined by processing said acceleration measurements using a Kalman filter.

23. The method of claim 19, further comprising providing corrective measurements in connection with horizontal motions of a head unrelated to body movement, wherein said corrective measurements supplement said acceleration measurements.

24. The method of claim 19, further comprising providing corrective measurements in connection with vertical motions of a head unrelated to body movement, wherein said corrective measurements supplement said acceleration measurements.

25. The method of claim 19, wherein said nominal gait pattern is derived by evaluating said stored gait feature statistics.

26. The method of claim 19, further comprising providing notification of said detected deviation in said nominal gait pattern.

27. A system for accumulating gait feature statistics to derive a nominal gait pattern of an individual, comprising:
a multi-axis accelerometer;
a processor coupled to said multi-axis accelerometer for analyzing measurements collected by said multi-axis accelerometer; and
an extraction component coupled to said processor for extracting current gait features associated with measurements analyzed by said processor.
